(19) United States
(12) Patent Application Publication
(10) Pub. No.: US 2010/0010046 A1
(43) Pub. Date: Jan. 14, 2010

(54) PHARMACEUTICAL FORMULATION FOR PARENTERAL ADMINISTRATION

(75) Inventors: Mark G. Currie, Sterling, MA (US); Daniel P. Zimmer, Somerville, MA (US)

Correspondence Address: HESLIN ROTHENBERG FARLEY & MESITI PC, 5 COLUMBIA CIRCLE, ALBANY, NY, 12203, US

(73) Assignee: IRONWOOD PHARMACEUTICALS, INC., Cambridge, MA (US)

(21) Appl. No.: 12/304,392

(22) PCT Filed: Jun. 8, 2007
(86) PCT No.: PCT/US07/70746

§ 371 Date: Jun. 19, 2009

Related U.S. Application Data
(60) Provisional application No. 60/804,478, filed on Jun. 12, 2006.

(51) Int. Cl. A61K 31/407; A61K 31/4439; A61P 25/00

(52) U.S. Cl. 514/338; 514/413

(57) ABSTRACT

Parenteral formulations comprising ketorolac, and a compound of formula II wherein R is hydrogen or lower alkyl. Such formulations are used for the treatment and prevention of pain.

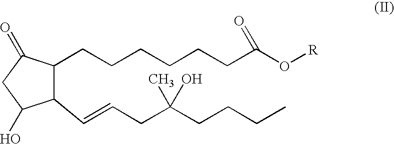

### CROSS REFERENCE TO RELATED APPLICATIONS

This application claims priority from co-pending provisional application Ser. No. 60/804,478, filed Jun. 12, 2006. The disclosures of this provisional application are incorporated by reference herein.

### FIELD OF THE INVENTION

The present invention relates to a parenteral pharmaceutical formulation comprising a mixture of ketorolac and a compound of formula II

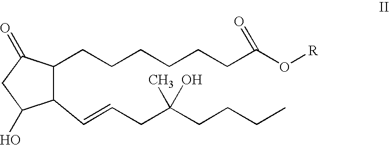

useful for treating pain.

### BACKGROUND OF THE INVENTION

Ketorolac (I),

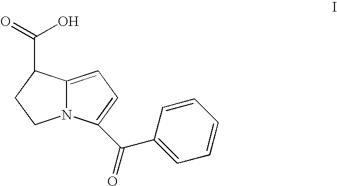

is a nonsteroidal anti-inflammatory drug (NSAID) administered orally, intravenously, and intramuscularly as its tromethamine salt. It is sold as a racemic mixture of (R) and (S) enantiomers and is used for moderate or severe pain management. Due to side effects, particularly ulcerogenic effects, ketorolac is only used on a short-term basis, generally not to exceed five days. Both the analgesic and the ulcerogenic activity have been shown to reside in (S)-ketorolac, as shown in formula Ia,

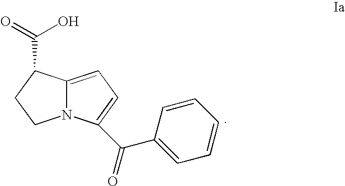

Misoprostol, a synthetic prostaglandin E₁ (PGE₁) analogue, has been shown to reduce and prevent ulcers. Misoprostol, sold as Cytotec®,

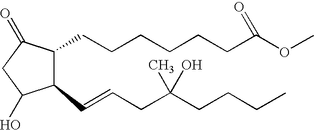

is a 1:1:1:1 mixture of 11R, 16S; 11S, 16R; 11R, 16R; and 11S, 16S isomers.

Oral compositions of NSAIDs, including ketorolac, together with a prostaglandin to reduce the potential for gastrointestinal side effects are known. However, misoprostol, a synthetic prostaglandin E₁ (PGE₁) analogue, is known to be chemically unstable at room temperature and breaks down in the presence of water (See U.S. Pat. No. 5,324,746). Arthrotec® which is a combined formulation of an NSAID (diclofenac) and misoprostol is an oral formulation.

### SUMMARY OF THE INVENTION

The invention relates to a parenteral pharmaceutical formulation comprising ketorolac and a compound of formula II,

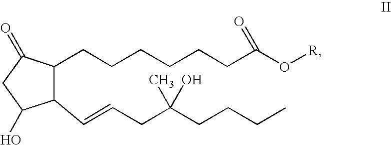

where R is hydrogen or lower alkyl. This parenteral formulation is useful for treating pain, including post-operative pain.

A further embodiment of the invention relates to a parenteral formulation comprising ketorolac, at least one proton pump inhibitor, and a compound of formula II,

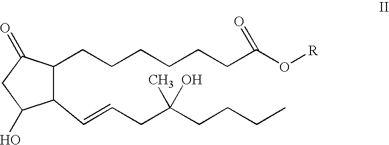

where R is hydrogen or lower alkyl.

The invention also relates to a method of treating and/or preventing pain via parenteral administration to a patient in need of such treatment, a therapeutically effective amount of ketorolac and a therapeutically effective amount of a compound of formula II. In addition, to treating and/or preventing pain, the methods of the invention may also mitigate one or more of inflammation, post-operative ileus, opioid-induced constipation, renal effects of ketorolac, and ulcerogenic effects of ketorolac.

The invention further relates to a process for making a lyophilized parenteral formulation comprising, consisting of, or consisting essentially of the steps of (a) adding ketorolac and a compound of formula II to lactose or HPMC and tertiary butyl alcohol wherein the tertiary butyl alcohol is present in an amount of from about 15% to about 33% volume/volume and whereby a formulation of ketorolac and compound II dispersed in lactose or HPMC is formed, (b) adjusting the pH of the formulation to between about 4 and about 5 with a citrate or acetate buffer, (c) freezing the formulation and (d) drying the formulation to obtain a moisture content of less than 1% by dry weight and a tertiary butyl alcohol content of less than 3% by dry weight. In certain embodiments, the citrate is sodium citrate. The invention further relates to the lyophilized parenteral formulation obtained by this processes.

### DETAILED DESCRIPTION OF THE INVENTION

Throughout this application, references are cited. The disclosure of these publications in their entireties is hereby incorporated by reference as if written herein. Also, terms and substituents are defined in the specification and retain their definition throughout.

For the purpose of this application, lower alkyl refers to alkyl groups of from 1 to 6 carbon atoms. Examples of lower alkyl groups include methyl, ethyl, propyl, isopropyl, butyl, s- and t-butyl and the like.

Also, as used herein, and as would be understood by the person of skill in the art, the recitation, “a compound”, is intended to include salts, solvates and inclusion complexes of that compound. The term “salt” refers to salts prepared from pharmaceutically acceptable non-toxic bases including inorganic and organic bases suitable pharmaceutically acceptable base addition salts for the compounds of the present invention include metallic salts made from aluminum, calcium, lithium, magnesium, potassium, sodium and zinc or organic salts made from lysine, N,N′-dibenzylethylenediamine, chloroprocaine, choline, diethanolamine, ethylenediamine, meglumine (N-methylglucamine), procaine, and tromethamine. In certain embodiments, the compound(s) of the invention is a tromethamine salt form.

Both ketorolac and compound II contain asymmetric centers, which give rise to enantiomers, diastereomers, and other stereoisomeric forms. Each chiral center may be defined, in terms of absolute stereochemistry, as (R)- or (S)-. The present invention is meant to include all such possible isomers, as well as, their racemic and optically pure forms. Optically active (R)- and (S)-, isomers may be prepared using chiral synthons or chiral reagents, or resolved using conventional techniques. All tautomeric forms are also intended to be included.

The graphic representations of racemic, ambiscalemic and scalemic or enantiomerically pure compounds used herein are taken from Maehr J. Chem. Ed. 62, 114-120 (1985): solid and broken wedges are used to denote the absolute configuration of a chiral element; wavy lines and single thin lines indicate disavowal of any stereochemical implication which the bond it represents could generate; solid and broken bold lines are geometric descriptors indicating the relative configuration shown but denoting racemic character; and wedge outlines and dotted or broken lines denote enantiomerically pure compounds of indeterminate absolute configuration.

The term “enantiomeric excess” is well known in the art and is defined for a resolution of ab into a+b as

$$e {e}_{a} = ( \frac{conc . of a - conc . of b}{conc . of a + conc . of b} ) \times 100$$

The term “enantiomeric excess” is related to the older term “optical purity” in that both are measures of the same phenomenon. The value of ee will be a number from 0 to 100, zero being racemic and 100 being pure, single enantiomer. A compound which in the past might have been called 98% optically pure is now more precisely described as 96% ee; in other words, a 90% ee reflects the presence of 95% of one enantiomer and 5% of the other in the material in question.

The term parenteral administration includes subcutaneous, intradermal, intramuscular, intravenous, intrathecal, and intraarticular administration.

The term “methods of treating or preventing” mean amelioration, prevention or relief from pain. The term “preventing” as used herein refers to administering a medicament beforehand to forestall or obtund an acute episode or, in the case of a chronic condition to diminish the likelihood or seriousness of the condition. The person of ordinary skill in the medical art (to which the present method claims are directed) recognizes that the term “prevent” is not an absolute term. In the medical art it is understood to refer to the prophylactic administration of a drug to substantially diminish the likelihood or seriousness of a condition, and this is the sense intended in applicants' claims. As used herein, reference to “treatment” of a patient is intended to include prophylaxis. Throughout this application, various references are referred to. The disclosures of these publications in their entireties are hereby incorporated by reference as if written herein.

Formulations of the present invention may also optionally include other therapeutic ingredients, preservatives, colorants, buffers, dyes, and the like. Any such optional ingredient must, of course, be compatible with the formulation of the invention to insure stability and must be added in appropriate amount. Such ingredients may include, ingredients which may effect solubility, such as sodium benzoate, ingredients which enhance patient comfort, as do substances added to make a solution isotonic, ingredients which enhance the chemical stability of a solution, as do antioxidants, inert gases, chelating agents, and buffers, and ingredients which protect a preparation against the growth of microorganisms, referring to all substances that act to retard or prevent the chemical, physical, or biological degradation of a preparation. Antimicrobial agents may include phenylmercuric nitrate and thimerosal, benzethonium chloride and benzalkonium chloride, phenol or cresol, or cholorobutanol.

The parenteral dose range for adult humans is generally from 10 to 100 mg of ketorolac and from 100 μg to 6 mg of compound II. The precise amount of formulation administered to a patient will be the responsibility of the attendant physician. However, the dose employed will depend on a number of factors, including the age and sex of the patient, the precise disorder being treated, and its severity.

The invention relates to a parenteral formulation comprising ketorolac and a compound of formula II

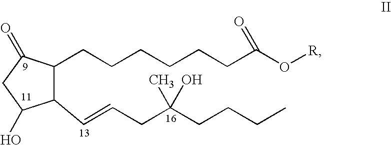

in which R is hydrogen or lower alkyl. A preferred lower alkyl is methyl.

In one embodiment ketorolac is racemic, in another embodiment ketorolac is >90% (S)-ketorolac, i.e. 80% ee. In another embodiment, either or both of ketorolac and the compound of formula II are in the form of a salt. In a preferred embodiment that salt is tromethamine.

In a further embodiment of the invention, a compound of formula II is a single isomer at C16:

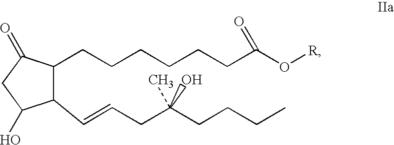

and more specifically formula IIb

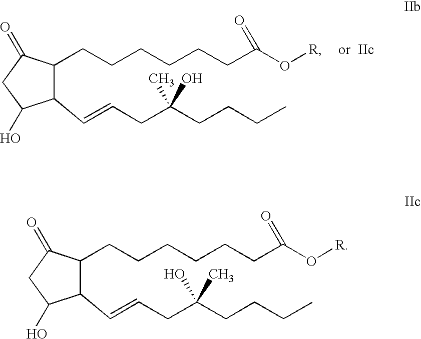

A further embodiment of the invention relates to a parenteral formulation of ketorolac and a compound of general formula II, wherein the hydroxyl at C11 is of a single chirality (either R or S) and the relative chiralities at C8 and C12 are fixed relative to C11 as shown in formula IId

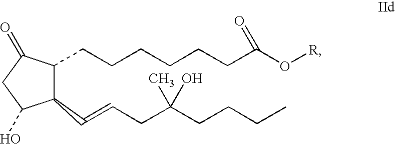

and more specifically, formula IIe,

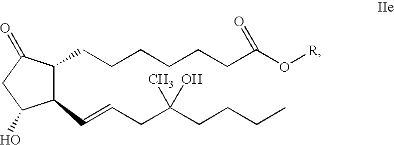

formula IIf,

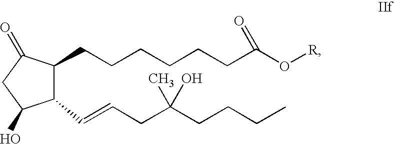

or formula IIg, which is a single isomer with all stereochemistry fixed:

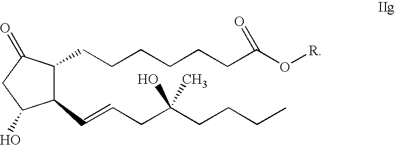

The invention also related to a method of treating or preventing pain comprising parenteral administration to a patient in need of such treatment, a therapeutically effective amount of ketorolac and a therapeutically effective amount of a compound of formula II. Such treatment may also mitigate one or more of: inflammation, post-operative ileus, opioid-induced constipation, NSAID renal effects, and ulcerogenic effects of ketorolac.

The invention also relates to a parenteral formulation comprising ketorolac, at least one proton pump inhibitor, and a compound of formula II. Examples of proton pump inhibitors include omeprazole, lansoprazole, rabeprazole, esomeprazole, and pantroprazole.

Advantages of the combination of ketorolac and compound II over ketorolac alone include one or more of: (1) better control of pain and inflammation, (2) reduction in incidence and/or severity of post-operative ileus, (3) reduced deleterious renal effects, (4) reduced ulcerogenic side effects, and (5) reduction of opioid-induced constipation in patients treated with opiates, ketorolac and compound II. Advantages of the combination of ketorolac and compound II over opioids for treatment of post-operative pain include one or more of: (1) reduction in incidence and/or severity of opioid-induced constipation, (2) avoidance of addiction and regulatory control issues and (3) absence of the development of tolerance to the analgesic effect of the medication.

Formulations for parenteral administration include aqueous and non-aqueous sterile injection solutions, which may contain anti-oxidants, buffers, bacteriostats and solutes which render the formulation isotonic with the blood of the intended recipient. Formulations for parenteral administration also include aqueous and non-aqueous sterile suspensions, which may include suspending agents and thickening agents. The formulations may be presented in unit-dose or multi-dose containers, for example sealed ampoules and vials, and may be stored in a freeze-dried (lyophilized) condition requiring only the addition of a sterile liquid carrier, for example saline, phosphate-buffered saline (PBS) or the like, immediately prior to use. Extemporaneous injection solutions and suspensions may be prepared from sterile powders of the kind described below.

One embodiment of the subject invention is a lyophilized ketorolac and misoprostol composition made from a bulk sterile filtered aqueous solution which contains 20% to 50% v/v tertiary butyl alcohol (TBA). Both the water and TBA are removed during the freeze-drying process. Residual water and TBA remaining after lyophilization are below 4% by weight of the dried lyophil. In one formulation, a vial dosage contains after completion of lyophilization: 10 mg to 100 mg of ketorolac as the tromethamine salt, 20 μg to 500 g of misoprostol, 2 mg to 200 mg of hydroxypropylmethyl cellulose (HPMC), and about 50 μg of sodium citrate. After reconstitution of this freeze-dried powder with 1.0 mL of either water for injection or bacteriostatic water for injection, a solution containing 20 μg/mL of misoprostol is obtained. The freeze-dried powder is packaged in a 5 mL vial and sealed with a lyophilization style closure within the freeze-dry chamber, and capped with an aluminum overseal. Initial rate kinetic analyses (i.e., monitoring the rate of formation of the major degradation product, as shown by formula III) are used to assess the chemical stability.

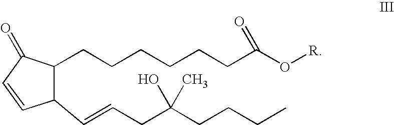

When the product is properly manufactured with the optimized formulation and process, the shelf-life should be greater than 12 months when the product is stored at 25° C. or less.

A minimum amount of buffer, commonly citrate buffer, is added to the formula to control pH. Typically, standard freeze-drying techniques are used to prepare the stabilized lyophil. An annealing technique may be used to decrease and more uniformly control the residual tertiary butyl alcohol in the freeze-dried product. Parameters that may affect product stability include the level of HPMC diluent present, the apparent pH of the lyophil, and the moisture content.

Lyophilization of a buffered HPMC formulation of ketorolac and compound II from a tertiary butyl alcohol (TBA)/water mixture provides improved product stability. The level of TBA which affords the product maximum stability ranges from 10-50% (v/v). The appropriate TBA level is selected to minimize the flammability potential and minimize the amount of TBA waste, which is generated after lyophilization without compromising stability or solubility.

The resulting residual TBA in the final product is below 3% of the cake weight to avoid toxicity concerns, and water is below 1% to maximize stability. Control of the freeze-dry cycle (both primary and secondary drying) is advantageous from the standpoint of commercial manufacture, and stability can be affected by processing parameters. Maximizing the amount of HPMC for a given amount of compound II will provide stability. For a formulation containing 20 μg/mL of compound II, the amount of HPMC can be about 200 mg.

Commonly the cake pH also affects product stability. Any pharmaceutically acceptable buffer can be employed, however, the citrate buffer is a common buffer for parenteral products. Since misoprostol is susceptible to both acid and base hydrolysis, it is probable that some buffer catalysis of the decomposition of compound II may occur. The amount of citrate buffer selected for the final formulation is chosen based on a compromise between sufficient buffer to adequately control pH and yet not itself significantly provide an alternate catalytic route.

The presence of moisture in the product will have a negative impact on product stability. It is therefore preferred that the formulation have the level of moisture as low as possible during the processing and to maintain that level throughout the shelf life of the product.

Typically, the ketorolac and compound II in HPMC formulation is freeze-dried using standard techniques. An annealing process may be used to reduce the residual tertiary butyl alcohol. In an annealing process the initial stage of the freeze drying process is carried out by freezing the compound II formulation to about −50° C., warming it to about −25° C., for about 2 hours, then refreezing it to about −5° C. Next, the freeze drying is continued to obtain a moisture content of less than 1% by dry weight and a tertiary butyl alcohol content of less than 3% by dry weight.

The potency of misoprostol has been demonstrated in vivo in dogs and has been shown to have a histamine stimulating gastric activity in gastric fistula. Similarly, rat models have also demonstrated a reduced lesion formulation of up to 60% by subcutaneous misoprostol dosage of 50 μg/kg of compound II in indomethacin-induced rat gastric ulcers.

Concentrations required to inhibit 50% of maximal histamine-stimulated acid secretion in an isolated canine parietal cell preparation is shown in Table 1 below.

TABLE 1

| COMPOUND | IC₅₀ (nM) |
|---|---|
| II, R═CH₃ | 3.8 |
| (11R,16R) + (11S,16S) + (11R,16S) + | |
| (11S,16R) | |
| II, R═CH₃ 11R,16S | 1.4 |
| II, R═CH₃ 11S,16R | >1000 |
| II, R═H (11R,16R) + (11S,16S) + | 2.8 |
| (11R,16S) + (11S,16R) | |

Techniques for the solid dispersion of misoprostol in HPMC and polyvinylpyrrolidone are known. (See U.S. Pat. No. 4,301,146). Other stabilization methods are described in U.S. Pat. No. 5,935,939, which includes microcrystalline and amorphous excipients chosen from hydroxypropyl cellulose, methyl cellulose, carboxymethyl cellulose, sodium carboxymethyl cellulose, cellulose acetate phthalate, cellulose acetate butyrate, hydroxyethyl cellulose, ethyl cellulose, polyvinyl alcohol, polyethylene glycol, starch polypropylene, dextrans, dextrins, hydroxypropyl β-cyclodextrin chitosan, co-(lactic/glycolic)copolymers, poly(orthoester), polyvinyl chloride, polyvinyl acetate, ethylene vinyl acetate, lectins, carbopols, silicon elastomers, cyclodextrins, polyacrylic polymers, maltodextrins, lactose, fructose, inositol, trehalose, maltose, and raffinose, (and other mono-, di- and tri-saccharides) and α-, β- and γ-cyclodextrins, preferably dextran, maltodextrin, hydroxypropyl β-cyclodextrin and maltose. (See U.S. Pat. No. 5,741,523).

In one embodiment, the formulation may be lyophilized in a single-dose, dual chamber cartridge intended to be used with an injection device. In certain embodiments thereof, one chamber of the cartridge contains the lyophil described above The other chamber contains about 1 mL of sterile 0.9% sodium chloride, optionally containing up to 20% v/v of ethanol. The injection device is used to reconstitute the sterile powder in one chamber with the sterile 0.9% sodium chloride in the other chamber. After reconstitution, the injection device is used to administer the injection. Alternatively, the lyophil may be dissolved in saline in a separate step or apparatus and subsequently introduced into a device for injection.

### EXAMPLES

### Example A

A second embodiment of the subject invention is a lyophilized ketorolac and compound II composition made from a bulk sterile filtered aqueous solution (1-8 mL) which contains 20% to 50% v/v alcohol (either TBA or ethanol). Both the water and alcohol are removed during the freeze-drying process. Residual water and alcohol remaining after lyophilization are below 4% by weight of the dried lyophil. In one formulation, a vial dosage contains after completion of lyophilization: 10 mg to 100 mg of ketorolac as the tromethamine salt, 20 μg to 1 mg of ester (formula IV) or free acid (formula V) as the tromethamine salt, 2 mg to 200 mg of hydroxypropylmethyl cellulose (HPMC), polyvinylpyrrolidone, cyclodextrin, succinic acid, or lactose, and citric acid to pH 3-6.

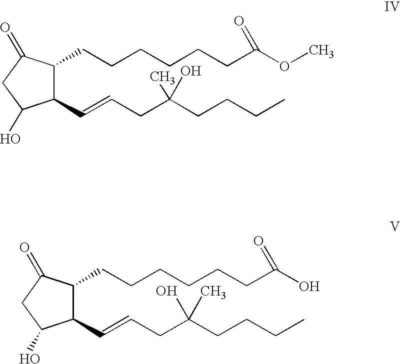

After reconstitution of this freeze-dried powder with 0.5-10 mL of either 0-200 mM phosphate buffer pH 7.4 for injection (containing 0-10% ethanol) or bacteriostatic phosphate buffer pH 7.4 for injection, a solution or suspension is obtained. The freeze-dried powder is packaged in a 2-10 mL vial and sealed with a lyophilization style closure within the freeze-dry chamber, and capped with an aluminum overseal.

### Example B

### HPMC Formulation of the Acid

A more specific embodiment of the subject invention is a lyophilized ketorolac and compound II composition made from a bulk sterile filtered aqueous solution (3 mL) which contains 20% v/v alcohol (ethanol). Both the water and alcohol are removed during the freeze-drying process. In one formulation, a vial dosage contains after completion of lyophilization: 30 mg of ketorolac as the tromethamine salt, 200 μg of free acid, formula V, 20 mg hydroxypropylmethyl cellulose (HPMC), and enough citric acid to bring the pH to 4.0. After reconstitution of this freeze-dried powder with 1 mL of 200 mM phosphate buffer pH 7.4 for injection or bacteriostatic phosphate buffer pH 7.4 for injection, a solution or suspension is obtained. The freeze-dried powder is packaged in a 5 mL vial and sealed with a lyophilization style closure within the freeze-dry chamber, and capped with an aluminum overseal.

### Example C

Another specific embodiment of the subject invention is a lyophilized ketorolac and compound II composition made from a bulk sterile filtered aqueous solution (3 mL) which contains 20% v/v alcohol (tertiary butyl alcohol). Both the water and alcohol are removed during the freeze-drying process. In one formulation, a vial dosage contains after completion of lyophilization: 30 mg of ketorolac as the tromethamine salt, 200 μg of free acid, formula V, 100 mg lactose, and citric acid to bring the pH to 4.0. After reconstitution of this freeze-dried powder with 1 mL of 200 mM phosphate buffer pH 7.4 for injection or bacteriostatic phosphate buffer pH 7.4 for injection, a solution or suspension is obtained. The freeze-dried powder is packaged in a 5 mL vial and sealed with a lyophilization style closure within the freeze-dry chamber, and capped with an aluminum overseal.

### In Vivo Models

### Rat Model of Postoperative Ileus:

Female CD rats are used to test the effect of test articles on delayed transit induced by abdominal surgery and manual manipulation of the small intestine. Groups of at least nine rats undergo abdominal surgery under isoflurane anesthesia. Surgery consists of laparotomy and 5 minutes of gentle manual intestinal massage. Following recovery from anesthesia, rats are dosed orally with either test article or vehicle (20 mM Tris) in a volume of 300 μl. 1 hour after dosing, intestinal transit rate is measured. Animals are again dosed with 300 μl of the test article followed immediately by 500 μl of a charcoal meal (10% charcoal, 10% gum arabic in water). To calculate the distance of the small intestine traveled by the charcoal front, after 20 minutes, the total length of the intestine as well as the distance traveled from the stomach to the charcoal front are measured for each animal.

### Rat Model of Opioid-induced Constipation:

Female CD rats are used to test the effect of test articles on delayed transit induced by opiate treatment. Animals are dosed with morphine at 2.5 mg/kg via intraperitoneal injection. Thirty minutes later test article is administered parenterally or orally. After ten minutes, animals are dosed with 0.5 mL of a charcoal meal (10% charcoal, 10% gum arabic in water). After 10 minutes, the total length of the intestine, as well as the distance traveled from the stomach to the charcoal front, are measured for each animal.

### Animal Models For Assessing Anti-Inflammatory and Analgesic Activity:

Any of a variety of animal models can be used to test the compounds of the invention for their effectiveness in reducing inflammation and treating pain. Useful compounds can exhibit effectiveness in reducing inflammation or pain in one or more animal models.

### Carrageenan-Induced Foot Pad Edema Model:

The model is described, for example, by Winter et al. (1962 Proc Soc Exp Biol Med 111:544). Briefly, rats are fasted with free access to water for 17 to 19 h before oral treatment with up to three doses of a test article, indomethacin or celecoxib, or a control vehicle (1% methylcellulose in deionized water). One hour after the last treatment, paw edema is induced by injecting 0.05 mL of a 2% carrageenan solution into the left hindpaw). The left hindpaw volume of each rat is measured using a plethysmometer before oral treatment, at the time of carrageenan injection and at 1.5 h, 3 h, 4.5 h after the injection of carrageenan. The edema volume of each rat at each time point is expressed as the change from the volume at the time of oral treatment and the anti-inflammatory effect in treated groups is expressed as % inhibition compared to the vehicle only group 1.5 h, 3 h and 4.5 h after the carrageenan injection. The significance of the difference between in edema different groups is assessed by a one-way analysis of variance (ANOVA) followed by the non-paired Dunnett t test. In this model, hyperalgesic response and PGE₂ production can also be measured (Zhang et al. 1997 J Pharmacol and Exp Therap 283:1069).

### Carrageenan-Induced Thermal Hyperalgesia:

This model is described by Hargreaves et al. (1988 Pain 32:77). Briefly, inflammation is induced by subplantar injection of a 2% carrageenan suspension (0.1 mL) into the right hindpaw. Three hours later, the nociceptive threshold is evaluated using a thermal nociceptive stimulation (plantar test). A light beam (44% of the maximal intensity) is focused beneath the hindpaw and the thermal nociceptive threshold is evaluated by the paw flick reaction latency (cut-off time: 30 sec). The pain threshold is measured in ipsilateral (inflamed) and in contralateral (control) hindpaws, 1 h after the oral treatment with the test compound or a control. The results can be expressed as the nociceptive threshold in seconds (sec) for each hindpaw and the percentage of variation of the nociceptive threshold (mean±SEM) for each rat from the mean value of the vehicle group. A comparison of the nociceptive threshold between the inflamed paw and the control paw of the vehicle-treated group is performed using a Student's t test, a statistically significant difference is considered for P<0.05. Statistical significance between the treated groups and the vehicle group is determined by a Dunnett's test using the residual variance after a one-way analysis of variance (P<0.05) using SigmaStat Software.

### Animal Models for Assessing Ulcers and Lesions in Ketorolac-Treated Rats

### Gastric Toxicity (1994 Proc Natl Acad Sci USA. 91:12013-7).

Animals are fasted for 16 hr prior to administration by gavage of either indomethacin or test article. Five hours after administration of doses, the animals are killed by CO2 and the stomachs are removed, excised along the greater curvature, and inspected for evidence of gastric glandular mucosal damage with a stereomicroscope. ED50 is defined as the dose at which 50% of the animals show evidence of gastric damage by visual inspection.

### Intestinal Toxicity (1994 Proc Natl Acad Sci USA. 91:12013-7).

Fed rats are dosed once with a suspension of vehicle (0.5% methylcellulose in water or drug (indomethacin 1-30 mg/kg or test article, intragastrically). After 72 hr, the animals are euthanized by CO2 asphyxiation, the abdominal cavity of each is opened, and the presence or absence of adhesions is noted by an investigator. The data are expressed as ED50, defined as dose resulting in 50% of animals with lesions (n=3-12).

1. A parenteral formulation comprising;
ketorolac, and;
a compound of formula II, 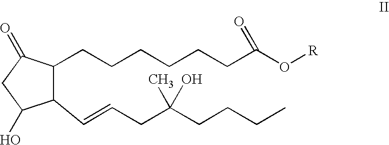
wherein R is hydrogen or lower alkyl.

2. A parenteral formulation according to claim 1, wherein R is hydrogen or methyl.

3. (canceled)

4. A parenteral formulation according to claim 2, wherein the compound of formula II is

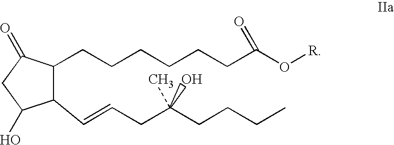

5. A parenteral formulation according to claim 4, wherein the compound of formula II is

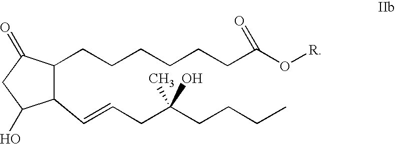

6. A parenteral formulation according to claim 4, wherein the compound of formula II is

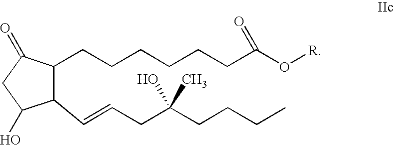

7. A parenteral formulation according to claim 2, wherein the compound of formula II is

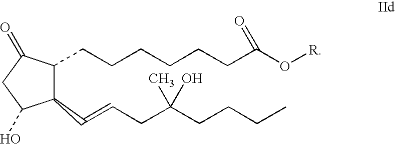

8. A parenteral formulation according to claim 7, wherein the compound of formula II is

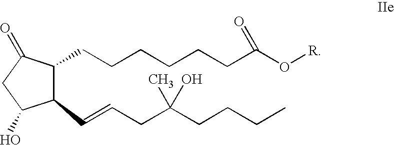

9. A parenteral formulation according to claim 7, wherein the compound of formula II is

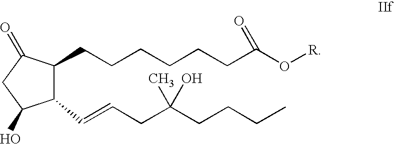

10. A parenteral formulation according to claim 7, wherein the compound of formula II is

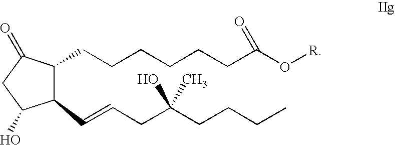

11. A parenteral formulation according to claim 1, wherein ketorolac is racemic.

12. (canceled)

13. A parenteral formulation according to claim 1, wherein ketorolac is >90% (S)-ketorolac.

14. (canceled)

15. A parenteral formulation according to claim 1, wherein one or both of ketorolac and a compound of formula II is in the form of a salt.

16. (canceled)

17. A parenteral formulation according to claim 15, wherein said salt is a tromethamine salt.

18. (canceled)

19. A parenteral formulation according to claim 1, further comprising at least one proton pump inhibitor.

20. (canceled)

21. A parenteral formulation according claim 19 wherein said proton pump inhibitor is chosen from omeprazole, lansoprazole, rabeprazole, esomeprazole, pantoprazole and omeprazole.

22. (canceled)

23. (canceled)

24. (canceled)

25. (canceled)

26. (canceled)

27. (canceled)

28. (canceled)

29. (canceled)

30. (canceled)

31. A method of treating or preventing pain comprising parenterally administering to a patient in need of such treatment, a therapeutically effective amount of ketorolac and a therapeutically effective amount of a compound of formula II

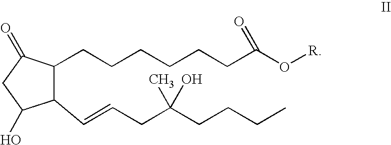

32. A method of treating or preventing pain comprising parentally administering to a patient in need of such treatment, a therapeutically effective amount of a parenteral formulation according to claim 1.

33. (canceled)

34. A method of treating or preventing pain while mitigating inflammation comprising parenterally administering to a patient in need of such treatment, a therapeutically effective amount of a parenteral formulation according to claim 1.

35. A method of treating or preventing pain while mitigating post-operative ileus comprising parenterally administering to a patient in need of such treatment, a therapeutically effective amount of a parenteral formulation according to claim 1.

36. A method of treating or preventing pain while mitigating opioid-induced constipation comprising parenterally administering to a patient in need of such treatment, a therapeutically effective amount of a parenteral formulation according to claim 1.

37. A method of treating or preventing pain while mitigating renal side effects comprising parenterally administering to a patient in need of such treatment, a therapeutically effective amount of a parenteral formulation according to claim 1.

38. A method of treating or preventing pain while mitigating ulcerogenic effects comprising parenterally administering to a patient in need of such treatment, a therapeutically effective amount of a parenteral formulation according to claim 1.

39. (canceled)

40. (canceled)

41. (canceled)

42. (canceled)

43. (canceled)

44. A process for making a lyophilized parenteral formulation comprising:
(a) adding ketorolac and a compound of formula II to lactose or HPMC and tertiary butyl alcohol, wherein said tertiary butyl alcohol is present in an amount of from about 15% to about 33% volume/volume whereby a formulation of ketorolac and compound II dispersed in lactose or HPMC is formed;
(b) adjusting the pH of said formulation to between about 4 and about 5 with a citrate or acetate buffer;
(c) freezing said formulation; and
(d) drying said formulation to obtain a moisture content of less than 1% by dry weight and a tertiary butyl alcohol content of less than 3% by dry weight.

45. The process of claim 44 wherein said buffer of step b) is sodium citrate.

46. The lyophilized parenteral formulation obtained in the process of claim 44.
